(19) United States
(12) Patent Application Publication
(10) Pub. No.: US 2010/0016244 A1
(43) Pub. Date: Jan. 21, 2010

(54) D-GLUCOPYRANOSE 1-[3,5-BIS (1,1-DIMETHYLETHY)-4-HYDROXYBENZOATE] AND ITS DERIVATIVES, PREPARATION AND USE THEREOF

(76) Inventor: Robert Vachy, Versailles (FR)

Correspondence Address: BROWDY AND NEIMARK, P.L.L.C.;624 NINTH STREET, NW, SUITE 300, WASHINGTON, DC, 20001-5303, US

(21) Appl. No.: 12/306,330

(22) PCT Filed: Jun. 23, 2006
(86) PCT No.: PCT/FR2006/001486

§ 371 Date: Mar. 17, 2009

(51) Int. Cl. A61K 31/7012; C07H 15/06; A61P 31/12

(52) U.S. Cl. 514/25; 536/18.2

(57) ABSTRACT

The invention relates to a D-glucopyranose 1-[3,5-bis(1,1-dimethyl-ethyl)-4-hydroxybenzoate compound defined by formula (I). It applies in particular to the preparation and the use of this compound and of its derivatives for the preparation of medicaments for the treatment and/or prevention of infections with enveloped viruses, and in particular, in humans, the herpes virus, the Aids virus, the flu virus, the hepatitis B virus, the hepatitis C virus, the dengue virus and the ebola virus, and, in animals, the porcine pseudorabies virus, for example.

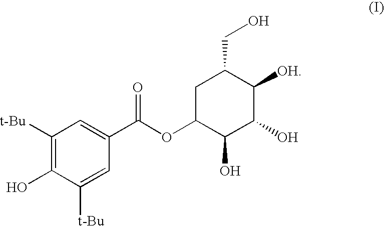

The present invention relates to a compound: D-glucopyranose 1-[3,5-bis (1,1-dimethylethyl)-4-hydroxybenzoate] and its derivatives. It applies particularly but not exclusively, to the preparation and the use of these compounds for preparing medecine to treat and/or prevent infections by enveloped-viruses, particularly in humans, such as herpes, AIDS, influenza of the hepatitis B and C, virus of Dengue, Ebola and, in animals, Aujewsky's disease as for instance Aujewsky's in pigs.

The action of these derivatives is unique. They are not blocking viral replication as virustatics but they are shredding the viral lipid-protein membrane. These derivatives are virucide.

The herpes and AIDS viruses, like many others (influenza of the hepatitis B and & C, SARS, Ebola etc. . . . ) are viruses surrounded by a lipid envelope unlike others—such as the virus of poliomyelitis who has no membrane—thus called naked-virus.

Enveloped-virus or naked-virus are non-cellular organism that are totally dependent of the cell they parasite for their survival. Viruses have no energy generating system (ATP) and no protein synthesis machinery. Although viral nucleic acids code for proteins, the synthesis of those proteins is performed on the host cell's ribosome. Hence, viruses must use the metabolic pathways of the cell as well as the capacity of these synthetic chemical factories that are the ribosome.

By rending impossible the access to viral metabolic pathways, virustatic (Tritherapy) disrupt metabolic pathways of the parasited molecules that the virus uses. This better reflects the poor tolerance of these biological therapies that block viral replication without killing the virus. Thus this limits significantly its effectiveness and use.

Taking into account the parasitic characteristics of the virus that makes it unable to survive outside a living eukaryotic cell, this invention seeks to prohibit its penetration into the living eukaryotic cell. Two methods are therefore possible:
Hiding the binding site of the host cell,
Eliminating the lipid envelope of the virus that contains the routing system and the protein adsorption on the membrane of the host cell.

In the first case, there is a risk of disruption of the metabolic external flux of the host cell, while lysing the viral envelope brings several benefits. It tends to annihilate skinned alive virus, making it unable to recognise the binding site and more importantly, it eliminates the proteins responsible for the adsorption of the virus on the membrane of the host cell. The virus and the cell can not merge, the virus left outside the cell dies.

It dies without any interference on the viral genome, in a way, by a mechanical action, limiting the risk of viral mutations which arise contrariwise to the mode of action of the virustatic.

This indifference towards virus' genetic heritage explains the effectiveness of these virucides on resistant mutant viruses to various new virucides available on the market.

### Mode of Action

The mode of action of virucides having a structure of di-tert-butyl such as BHT (butylhydroxytoluene) has been demonstrated in clinical trials against double-blind placebo in humans, by the disappearance or abortion of the herpes simply by application of a topical medicine from the onset.

Unlike the molecules acting on DNA, which induce a growth slow down, BHT is not involved in viral synthesis. One should seeks the origin of the properties of BHT elsewhere, in fact, Brugha M Jr, in an article published in “Science”, demonstrated two points:
first, that chickens receiving food containing 200 ppm BHT were protected against infection inoculated by the virus responsible of the Newcastle disease (VMN). He noted a decrease in sero-conversion proportional to the administered BHT dose. Extending its experiment with cultures of pre-treated embryonic chicken cells with 25 μg/ml BHT, he discovered that virus production is reduced by 65%.
second, that BHT inhibited the development of RNA virus (VMN) as well as that the development of DNA virus (VHS). He mentioned as a reason for this effect, a possible alteration of the envelope of the virus by the hydrophobic properties of BHT, although the effect of agonist VMN on the aggregation of chicken's erythrocytes—known characteristic of the membrane of this virion—is not changed, which seemed to him contradictory.

This hypothesis also proposed by Reimund and Cupp suggests that a modification of the geometry of the virus' lipid envelopes should prevent them to bind the membrane of the host cell.

Using electron microscopy, WINSTON, however, highlights the alteration, or even the break, of the virus' lipid envelopes, under the effect of treatment with BHT. BAMFORD demonstrates that the alteration of the viral envelope leads to the elimination of a protein (P3) responsible of the adsorption of the virus on the membrane of the host cell.

It remained to demonstrate the physico-chemical mechanism of these reactions.

Studying by electronic spin resonance, the composition of lipid envelopes, Aloia reveals the fluidity of enveloped-virus' membrane and in particular of HIV's membrane, under the effect of heat or BHT. By changing the composition of lipid envelopes and the cholesterol/phospholipid ratio, the BHT reduces the membrane rigidity by disrupting its structure. This disruption, coupled with the loss of adsorption ability, prevents any recognition and any binding of the virus on the membrane of the host cell. ALOIA experimentally confirm that 30 minutes incubation at 37° C. in 320 μg/ml BHT causes a decrease in viral infectivity on H9 lymphocytes, by a logarithmic factor of 4.

With AVF1 (3.5-di-tert-butyl-4-hydroxybenzoate octa-oxy-ethylene glycol), a substance derived from BHT, one manages to decrease HIV's infectivity by 7 log.

In summary, BHT's mode of action is complex:
virucidal, lysis of the protein-lipid envelope is explained by the hydrophobic properties of BHT. By promoting the binding with the transmembrane protein of the viral envelope they induce a modification of the cholesterol/phospholipid ratio responsible of the structural disruption of the envelope, its dehiscence and the expulsion of the viral adsorption protein.
fusion-inhibitor through inability to identify and to merge on the cellular binding site.

Without cytopathic action on cells at effective doses, BHT is non-toxic for the organism, it only targets the membrane encoded by the virus and not the one of the host cell.

Through these complex reactions, viruses and membrane are no longer compatible. Key and lock being changed, the virus can not open the doors of the host cell for its reproduction. It dies being phagocyted.

The lipophilic properties of BHT and its specific mode of action, precise and limited, led to think that the group di-phenyl-tert-butyl may play a predominant role. It was therefore imperative for us to increase the availability of the molecule without altering its structure.

For this purpose, the invention proposes the preparation of compound D-glucopyranose 1-[3,5-bis (1,1-dimethylethyl)-4-hydroxybenzoate] defined by the following formula:

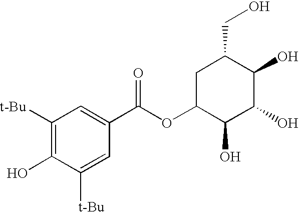

The process of preparation of the compound D-glucopyranose 1-[3,5-bis (1,1-dimethylethyl)-4-hydroxybenzoate] comprises the following steps:
the production of the chloride of the 3,5-di-t-butyl-4-hydroxybenzoic acid,
a esterification by the reaction of the obtained chloride acid and the D-glucopyranose.

The compound according to the invention and its potential derivatives and additional salts to a mineral or organic acid pharmaceutically acceptable may be presented in a composition consisting of at least a pharmaceutically acceptable carrier.

This composition may arise for instance as tablets, capsules, dragees, drinkable solutions or suspensions, emulsions, suppositories.

In addition to non-toxic and pharmaceutically acceptable inert excipients, such as distilled water, glucose, lactose from starch, talc, vegetable oils, ethylene glycol . . . , the compositions thus obtained can also contain preservation agents.

Other active ingredients may be added to these compositions such as 3,5-di-t-butyl-4-hydroxybenoic acid (BG4) or 3.5-di-tert-butyl-4-hydroxybenzoate octa-oxy-ethylene glycol (AVF1) or a pharmaceutically acceptable derivatives.

The amount of compound according to the invention and any other active ingredients in such compositions will vary depending on the application, age and weight of the patient.

The synthesis of 3,5-di-t-butyl-4-hydroxybenoic acid (BG4), and its halides, such as chloride and bromide, was described in the application EP 0 269 981.

This acid has been proposed for the preparation of antiviral drugs for the treatment of diseases linked to infection of a person by viruses having a lipid envelope and especially the herpes virus, or AIDS.

The compound of the present invention has several advantages particularly with regard to the BHT and 3,5-di-t-butyl-4-hydroxybenoic acid (BG4):
Better solubility in water which facilitates the development of pharmaceutical preparations for a more suitable product,
Virucidal activity in lower concentrations,
A pro drugs effect.

An example of preparing a compound according to the invention will be described below, as a non-limiting example.

The process of preparing about one kilogramme of the compound D-glucopyranose 1-[3,5-bis (1,1-dimethylethyl)-4-hydroxybenzoate comprises the following steps:

The first step comprises the synthesis of acid chloride

In a flask, 700 grams of 3,5-di-t-butyl-4-hydroxybenoic acid are dissolved while stirring, in 1400 ml of dioxane. Then, 450 grams of thionyl chloride (3 equivalents) are introduced and the mixture is heated to 80° C. for 3 hours.

The progress of the reaction is monitored by thin layer chromatography (TLC). Once the reaction is completed, the excess of thionyl chloride is removed by evaporation under vacuum and then the mixture is incorporated in 1400 ml of dioxane.

The second step comprises an esterification

In a flask, 360 grams of D-glucopyranose are dissolved in 500 ml of dioxane, then 170 ml of pyridine are added.

The solution obtained during the first step is fed into the flask and then the mixture is shaken at 50° C. for 3 hours.

The progress of the reaction is monitored by thin layer chromatography (TLC), the reference front or RF is 0.05 using a mixture toluene/formic acid/acetone and phthalate para-anisidine as a developer,

Once the reaction is completed, solvents are eliminated by evaporation under vacuum.

Then the gross product is dissolved in a mixture of water/ethyl acetate (to a total of 10 liters). After settling and washing the organic phase with acidic water, the latter is concentrated. The product thus obtained is recrystallized by a mixture of ethanol/water mixture (20 liters) and then filtered on frit and dried.

The compound RDW031 of a molecular weight of 412.54 g·mol-1 is obtained with a purity of 98% controlled by liquid chromatography (HPLC) and further characterized by proton NMR at 400 MHz in deuterated chloroform.

The compound RDW031 of the present invention has several advantages over BHT and the 3,5-di-t-butyl-4-hydroxybenoic acid (BG4):
a better water solubility which facilitates the development of pharmaceutical preparations best suited for a drug.

| | BG4 | RDW031 |
|---|---|---|
| Solubility ½ H | 0.84 g/litre (no | 1.08 g/litre (no |
| at 100° C. | desolubilization at | desolubilization at |
| | room temperature | room temperature |
| Solubilité ½ H | No measurable | 40 mg/litre |
| à 23° c. | | |

### Test No 2

| | BG4 | RDW031 batch RV 34 |
|---|---|---|
| Solubility 12 H | insoluble | 1.2 g/litre |
| at 23° C. | | |
| Materiel and | | 250 mg (slight excès) of RDW031 batch |
| methode | | RV41 + 100 ml H₂O stirred for 48 hours. |
| | | This gives a suspension which is then filtered |
| | | and concentrated under vacuum and |
| | | weighted. |

### Test no 3

| | BG4 | RDW031 batch RV 41 |
|---|---|---|
| Solubility | 0.13 g/litre | 1.23 g/litre |
| after | pH = 5.6 | Note: formation of a fine white |
| stirring for | | suspension |
| 48 H at | | →centrifugation |
| 23° C. | | pH = 5.9 |
| Materiel | 1 g (excès of BG4, | 1 g (exces of RDW31 batch |
| and | originated from SIGMA- | RV41) + 100 ml H₂O stirred for |
| methode | ALDRICH) + 100 ml H₂O | 48 hours. This gives a |
| | stirred for 48 hours. This | suspension which is then |
| | gives a suspension which is | filtered, as the trouble |
| | then filtered. The filtrate is | persists, the suspension is |
| | then evaporated under | centrifuged and the supernatant |
| | vacuum and weighted | is then evaporated and weighted |

A virucidal activity at very low concentrations
A pro-drug effect: D-glucopyranose 1-[3,5-bis (1,1-dimethylethyl)-4-hydroxybenzoate] and 3,5-di-t-butyl-4-hydroxybenzoic acid, structure that decomposes, forming a highly active equilibrium, the two molecules having a strong virucidal power (reduced by 5 log the virulence of a HIV culture)

### Virologic Studies on VHS (Herpes Simplex Virus)

Results of the tests conducted in the laboratory of Prof. Chiron, (Faculty of Pharmacy of Tours):

Solution at 0.946 g/l dans l'eau

| RDW 031 | | pur | ½ | ⅕ |
|---|---|---|---|---|
| | mg/ml | 0.85140 | 0.42570 | 0.17028 |
| | Contact time | | | |
| test n° 4 | 15 min | 1.25 | — | 0.00 |
| code: 04/179 | 30 min | 1.86 | — | 0.00 |
| 0.946 g/l (water) | 60 min | 2.15 | 0.00 | 0.00 |
| | 120 min | 2.32 | 1.48 | 0.00 |

Decrease expressed in log

In the above example, RDW031's virucidal activity on herpes begins of concentration much lower (0009%) than the one required for the effectiveness of BG 4 on VHS (0.5%) (FR 2 668 931)

### RDW 031

### Concentrations to Study

hypothesis: 10 mg/8 ml (solubility check)
Stock-solution: 33.47 mg/24.10 ml (x 1.11 C) i.e.: 11.11 mg/8 ml

| Dilutions | Pur | ½ | ⅕ | 1/20 |
|---|---|---|---|---|
| Mg/ml | 1.25000 | 0.62500 | 0.25000 | 0.06250 |
| Contact time | | | | |
| 15 min | — | 3.68 | 0.00 | — |
| 30 min | — | — | — | 0.21 |
| 60 min | — | — | — | — |
| 120 min | — | — | — | — |
| Dilutions | 1/50 | 1/200 | 1/500 | 1/1000 |
| Mg/ml | 0.02500 | 0.00625 | 0.00250 | 0.00125 |
| Contact time | | | | |
| 15 min | — | — | — | — |
| 30 min | 0.31 | — | — | — |
| 60 min | — | 1.06 | 1.06 | — |
| 120 min | — | — | 1.15 | 0.31 |

Reduction expressed in log

Expressed in Mol, the comparisons are in favor of the new molecule RDW 031, which acts at concentrations inferior to a log for a substantially identical inhibitory activity:
BG 4: from 0.5% to 1%, i.e.: 0.04 to 0.02 Mol,
AVF1: from 0.5% to 1%, i.e.: 0.0083 at 0166 Mol (8.3×10⁻³ to 1.66×10⁻² Mol)
RDW031 active at concentration starting of 0.0625%, i.e.: 0.0015 Mol (1.5×10⁻³ Mol)

It is worth recalling that the BHT, which has a very low toxicity, thus remaining a reference molecule, act on enveloped-viruses only at concentrations of 8 to 10%, that is to say at molars concentrations of 0.3 to 0.4 Mol that are 100 times stronger than RDW 031.

Thus, the D-glucopyranose 1-[3,5-bis (1, 1-dimethylethyl)-4-hydroxybenzoate] is a new molecule that combines a better solubility, a greater virucidal activity at lower doses than those of BHT and BG4.

As these molecules, the hydrophilic pole leads to the disintegration of the viral envelope of the virus herpes simplex (VHS) and has no effect on polio virus (naked-virus).

Its activity concerns all enveloped-viruses and particularly the AIDS virus for which promising studies are underway for various pharmaceutical packaging: film-coated tablets for oral administration in combination or in substitution of protease inhibitors when they are poorly supported.

The very low cyto-toxicity and high therapeutic scope eases the use with children. Without interference on the viral and human genome, it is possible first-line medication in pregnant women. All studies on rats have never shown any detectable effects on progeny nor on mutagenic effect, as this is expected with active virucide without interference on the viral or human genome.

The invention is a serious step forward in the battle against enveloped-viruses and especially against AIDS. One can hope viruses eradication by disappearance of viral loads which is not accessible to current virustatic that block partially the viral replication without killing the virus.

The therapeutic failures force the proliferation of drug combinations.

Only virucide can totally eliminate the virus colonies and allow the revival of the white line of CD 4 lymphocytes in particular and to restore the immune system of the body that HIV paralysis.

At the end of the regulatory pharmaco-toxicological tests, studies on humans will began.

From now on clinical trials on avian and porcine influenza will be undertaken. They will guide future studies.

1. Compound D-glucopyranose 1-[3,5-bis (1,1-dimethylethyl)-4-hydroxybenzoate defined by the formula:

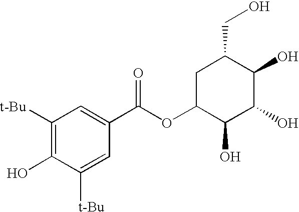

2. Compound of claim 1,
presenting as the form of derivatives or of addition salts to a mineral or organic acid pharmaceutically acceptable.

3. Composition,
comprising at least a compound D-glucopyranose 1-[3,5-bis (1,1-dimethylethyl)-4-hydroxybenzoate defined by the formula: 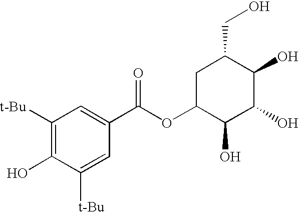
and at least one pharmaceutically acceptable excipient.

4. Composition,
comprising at least a compound D-glucopyranose 1-[3,5-bis (1,1-dimethylethyl)-4-hydroxybenzoate defined by the formula: 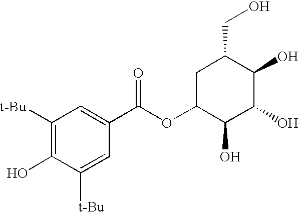
and at least 3,5-di-t-butyl-4-hydroxybenzoic acid or a pharmaceutically acceptable derivatives of 3,5-di-t-butyl-4-hydroxybenzoic acid and at least a pharmaceutically acceptable excipient.

5. Composition,
comprising at least a compound D-glucopyranose 1-[3,5-bis (1,1-dimethylethyl)-4-hydroxybenzoate defined by the formula: 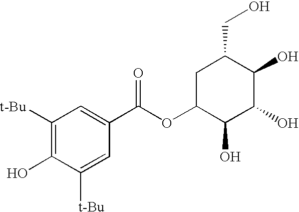
and at least 3.5-di-tert-butyl-4-hydroxybenzoate octa-oxy-ethylene glycol or derivatives pharmaceutically acceptable of 3,5-di-tert-butyl-4-hydroxybenzoate octa-oxy-ethylene glycol and at least a pharmaceutically acceptable excipient.

6. Composition according to claim 3, presenting as one of the following forms: tablets, capsules, dragees, drinkable solutions or suspensions, emulsions, suppositories.

7. A method for preparation of medicine for the treatment and/or preventing infections by enveloped-viruses using the compound D-glucopyranose 1-[3,5-bis (11-dimethylethyl)-4-hydroxybenzoate defined by the formula:

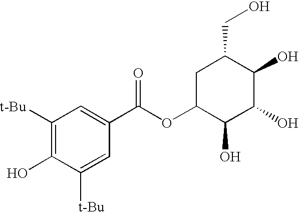

8. Composition according to claim 5, presenting as one of the following forms: tablets, capsules, dragees, drinkable, solutions or suspensions, emulsions, suppositories.

9. Composition according to claim 7, presenting as one of the following forms: tablets, capsules, dragees, drinkable, solutions or suspensions, emulsions, suppositories.
